(19) United States
(12) Patent Application Publication
(10) Pub. No.: US 2022/0058744 A1
(43) Pub. Date: Feb. 24, 2022

(54) DATA PROCESSING SYSTEMS AND METHODS FOR CALCULATING COST AND BENEFIT PARAMETERS

(71) Applicant: Visionworks of America, Inc., San Antonio, TX (US)

(72) Inventor: Murad Dar, San Antonio, TX (US)

(21) Appl. No.: 17/108,106

(22) Filed: Dec. 1, 2020

Related U.S. Application Data
(60) Provisional application No. 63/069,257, filed on Aug. 24, 2020.

(51) Int. Cl. G06Q 40/08; G06Q 10/10; G16H 40/20; G16H 10/60; G06Q 30/02; G06F 3/0482

(52) U.S. Cl. CPC G06Q 40/08; G06Q 10/10; G06F 3/0482; G16H 10/60; G06Q 30/0206; G16H 40/20

(57) ABSTRACT

Systems and methods for calculating cost and benefit parameters under an eye care plan during an eye care purchase transaction and presenting such costs and benefits to an eye care plan participant are disclosed. Data associated with a particular plan and participant is collected from various sources to determine the specific options available to the participant. A data structure is generated and updated to track the participant's selections during an eye care product and/or services transaction and maintain transaction data. The costs and benefits to the participant are dynamically updated and presented to the participant in a graphical user interface as the purchase process progresses so that the participant is fully informed of the costs and benefits during each phase of the process.

### CROSS-REFERENCE TO RELATED APPLICATIONS

This application claims priority to U.S. Provisional Patent Application Ser. No. 63/069,257, filed Aug. 24, 2020, entitled “DATA PROCESSING SYSTEMS AND METHODS FOR CALCULATING COST AND BENEFIT PARAMETERS,” which is hereby incorporated herein by reference in its entirety.

### BACKGROUND

Many people have vision insurance that at least partially covers the cost of annual eye exams and/or corrective lenses. Such insurance plans may have varying levels of coverage that depend on one or more options selected by the insured, such as, in the case of eyewear, the type of lenses and the type and/or brand of frames chosen by the insured individual. The total cost to the insured of corrective lenses under a vision plan may be widely variable because of the many options available but determining that cost during the eyewear purchasing process can be difficult and confusing. Typically the total cost of eyewear is presented only at the end of the eyewear purchasing process, which does not allow the insured to make informed decisions during the eyewear purchasing process to minimize the cost to the insured and/or maximize the benefits to the insured under the vision plan. This situation is complicated by the fact that the coverage provided by vision insurance plans can vary significantly from employer to employer, and the fact that many individuals aren't aware of the details of their individual vision insurance plans.

### SUMMARY

A data processing system for processing optical options, according to various embodiments, may include: at least one computer processor; an electronic communications transceiver; and memory operatively coupled to the at least one computer processors, wherein the at least one computer processor is adapted for: executing computer-readable instructions that, when executed by the at least one computer processor, facilitate the calculation of optical options based on user input, wherein the computer-readable instructions comprise instructions for: electronically communicating, by the electronic communications transceiver, with a plurality of optical option plan data repositories to identify and retrieve optical option data associated with a plurality of optical option plans; generating, by the at least one computer processor, an optical option plan data structure for each of the plurality of optical option plans; analyzing, by the at least one computer processor, the optical option data associated with each of the plurality of optical option plans to determine particular optical options for each of the plurality of optical option plans; populating, by the at least one computer processor, based at least in part on the analysis of the optical option data associated with each of the plurality of optical option plans, each optical option plan data structure with particular optical options associated with a respective optical option plan of the plurality of optical option plans; electronically receiving, by the electronic communications transceiver, a request to initiate an optical item interaction, the request comprising participant identification data and optical option plan identification data; at least partially in response to receiving the request: identifying, by the at least one computer processor, a particular optical option plan data structure based at least in part on the optical option plan identification data; generating, by the at least one computer processor, an interaction data structure; populating, by the at least one computer processor, one or more fields of the interaction data structure using the participant identification data and the optical option plan identification data; populating, by the at least one computer processor, an interaction status field in the interaction data structure with a first status; determining, by the at least one computer processor, a total available value based at least in part on the particular optical option plan data structure; and determining, by the at least one computer processor, a first plurality of optical options based at least in part on the particular optical option plan data structure; generating, by the at least one computer processor, a graphical user interface configured to: solicit a selection of an optical option from among the first plurality of optical options; display an indication of a respective participant cost for each option of the first plurality of optical options; display an indication of a respective benefit value for each option of the first plurality of optical options; and display an indication of a total available benefit value; electronically receiving, by the electronic communications transceiver, a selection of a first optical option from among the first plurality of optical options via the first graphical user interface; updating, by the at least one computer processor, the interaction status field in the interaction data structure based at least in part on the selection of the first optical option; and updating, by the at least one computer processor, one or more fields in the interaction data structure based at least in part on the first optical option.

In particular embodiments, one or more of the first plurality of optical options is selected from a group consisting of: (a) contact lenses; (b) glasses; (c) lenses; (d) frames; (e) lens coatings; and (f) an eye exam. In particular embodiments, a first subset of the plurality of optical option plan data repositories is associated with a first optical option plan provider; a second subset of the plurality of optical option plan data repositories is associated with a second optical option plan provider; and the first optical option plan provider is distinct from the second optical option plan provider. In particular embodiments, the computer-readable instructions further comprise instructions for: determining a benefit value for the first optical option based at least in part on the optical option plan data structure; determining a participant cost for the first optical option based at least in part on the optical option plan data structure; determining a remaining benefit value based at least in part on the total available benefit value and the benefit value for the first optical option; and updating the graphical user interface to: display the first optical option; display an indication of the benefit value for the first optical option; display an indication of the participant cost for the first optical option; and display an indication of the remaining benefit value. In particular embodiments, the total available benefit value is a total benefit value for a particular component of the selected optical option; and the particular component of the selected optical option is selected from a group consisting of: lenses; frames; and contact lenses. In particular embodiments, the computer-readable instructions further comprise instructions for updating the graphical user interface to display an indication of an interaction status based at least in part on the updated interaction status field in the interaction data structure.

A non-transitory computer-readable medium, according to various embodiments, may store computer-executable instructions for: receiving an indication that an individual has selected one or more vision products for purchase; at least partially in response to receiving the indication, accessing from one or more data stores, data regarding a vision plan held by the individual covering at least one of the one or more vision products; receiving a listing of the one or more selected vision products; and based at least in part on the received listing and the accessed vision plan data, communicating to the individual, via a computer interface: an amount of savings that they will receive if they complete a purchase of the one or more vision products; and an indication of benefits, under the vision plan, that will go unused if the individual completes the purchase without adding additional products to the purchase or substituting one or more of the vision products with another item.

In particular embodiments, accessing the data regarding the vision plan held by the individual comprises identifying the vision plan held by the individual from among a plurality of vision plans based on identification information associated with the individual. In particular embodiments, each plan of the plurality of vision plans is associated with a particular provider. In particular embodiments, a first one or more plans of the plurality of vision plans is associated with a first particular provider; a second one or more plans of the plurality of vision plans is associated with a second particular provider; and the first provider is distinct from the second provider. In particular embodiments, accessing the data regarding the vision plan held by the individual comprises identifying the vision plan from among a plurality of vision plans held by the individual. In particular embodiments, the computer-executable instructions may include instructions for identifying a second vision plan from among the plurality of vision plans held by the individual; accessing data regarding the second vision plan held by the individual covering at least one of the one or more vision products; and based at least in part on the received listing and the accessed second vision plan data, communicating to the individual, via the computer interface: a second amount of savings associated with a purchase of the one or more vision products under the second vision plan; and an indication of benefits, under the second vision plan, that will go unused if the individual completes the purchase of the one or more vision products under the second vision plan without adding additional products to a purchase transaction associated with the purchase of the one or more vision products under the second vision plan. In particular embodiments, the additional products comprise one or more products selected from a group consisting of: (a) a lens coating; (b) lens polarization; (c) lens light reactivity; (d) a particular lens thinness; and (e) lens magnification progressivity.

A computer-implemented data processing method for electronically calculating vision plan costs and benefits, according to various embodiments, may include: receiving, by one or more computer processors, participant data for a participant via the one or more participant data inputs; retrieving, by one or more computer processors from a data repository, based at least in part on the participant data, plan data for a particular vision plan; generating, by one or more computer processors, a transaction data structure for an eyewear product purchase transaction associated with the participant; populating, by one or more computer processors, one or more data fields in the transaction data structure using the plan data and the participant data; populating, by one or more computer processors, a transaction status field in the transaction data structure with a first status; determining, by one or more computer processors, based at least in part on the plan data, a total available benefit value; determining, by one or more computer processors, based at least in part on the plan data, a first plurality of eyewear product options; determining, by one or more computer processors, based at least in part on the plan data, a respective participant cost for each option of the first plurality of eyewear product options; determining, by one or more computer processors, based at least in part on the plan data, a respective benefit value for each option of the first plurality of eyewear product options; generating, by one or more computer processors on a video display unit, a first graphical user interface for receiving a selection of a first eyewear product option from among the first plurality of eyewear product options, wherein the first graphical user interface comprises: an indication of the respective participant cost for each option of the first plurality of eyewear product options; an indication of the respective benefit value for each option of the first plurality of eyewear product options; and an indication of the total available benefit value; receiving, by one or more computer processors via the first graphical user interface, the selection of the first eyewear product option; updating, by one or more computer processors, based at least in part on the selection of the first eyewear product option, the transaction status field in the transaction data structure with a second status; determining, by one or more computer processors, based at least in part on the selection of the first eyewear product option and the plan data, a participant cost for the first eyewear product option; determining, by one or more computer processors, based at least in part on the selection of the first eyewear product option and the plan data, an adjusted total available benefit value; determining, by one or more computer processors, based at least in part on the selection of the first eyewear product option and the plan data, a second plurality of eyewear product options; determining, by one or more computer processors, based at least in part on the plan data, a respective participant cost for each option of the second plurality of eyewear product options; determining, by one or more computer processors, based at least in part on the plan data, a respective benefit value for each option of the second plurality of eyewear product options; and generating, by one or more computer processors on a video display unit, a second graphical user interface for receiving a selection of a second eyewear product option from among the second plurality of eyewear product options, wherein the second graphical user interface comprises: an indication of the respective participant cost for each option of the second plurality of eyewear product options; an indication of the respective benefit value for each option of the second plurality of eyewear product options; an indication of the participant cost for the first eyewear product option; and an indication of the adjusted total available benefit value.

In particular embodiments, the method further comprises determining, by one or more computer processors, based at least in part on the plan data, a respective retail price for each option of the first plurality of eyewear product options; and the first graphical user interface further comprises an indication of the respective retail price for each option of the first plurality of eyewear product options. In particular embodiments, the second graphical user interface further comprises the indication of the total available benefit value. In particular embodiments, the method further comprises populating, by one or more computer processors, based at least in part on the selection of the first eyewear product option, one or more additional data fields in the transaction data structure. In particular embodiments, each of the first plurality of eyewear product options is a lens option and each of the second plurality of eyewear product options is a frame option. In particular embodiments, the method further comprises populating, by one or more computer processors, based at least in part on the selection of the first eyewear product option, a data field in the transaction data structure with a benefit value associated with the first eyewear product option. In particular embodiments, the method further comprises populating, by one or more computer processors, based at least in part on the selection of the first eyewear product option, a data field in the transaction data structure with a participant cost associated with the first eyewear product option.

A data processing system for processing cost and benefit data associated with a vision product purchase transaction, according to various embodiments, may include: at least one computer processor; and memory operatively coupled to the at least one computer processors, wherein the at least one computer processor is adapted for: executing computer-readable instructions that, when executed by the at least one computer processor, facilitate the calculation of vision plan benefits and costs based on user input, wherein the computer-readable instructions comprise instructions for: scanning a plurality of vision plan data repositories to identify vision plan data associated with a plurality of vision plans; generating a vision plan data structure for each of the plurality of vision plans; populating each vision plan data structure with vision plan data associated with a respective vision plan of the plurality of vision plans; receiving a request to initiate an eyewear product purchase, the request comprising participant identification data and plan identification data; at least partially in response to receiving the request: identifying a vision plan data structure based at least in part on the plan identification data; generating a transaction data structure; populating one or more fields of the transaction data structure using the participant identification data and the plan identification data; populating a transaction status field in the transaction data structure with a first status; determining a total available benefit value based at least in part on the identified vision plan data structure; and determining a first plurality of eyewear product options based at least in part on the identified vision plan data structure; generating a graphical user interface configured to: solicit a selection of an eyewear product option from among the first plurality of eyewear product options; display an indication of a respective participant cost for each option of the first plurality of eyewear product options; display an indication of a respective benefit value for each option of the first plurality of eyewear product options; and display an indication of a total available benefit value; detecting a selection of a first eyewear product option from among the first plurality of eyewear product options via the first graphical user interface; updating the transaction status field in the transaction data structure based at least in part on the selection of the first eyewear product option; and updating one or more fields in the transaction data structure based at least in part on the first eyewear product option.

In particular embodiments, one or more of the first plurality of eyewear product options is selected from a group consisting of: (a) contact lenses; (b) glasses; (c) lenses; (d) frames; (e) lens coatings; and (f) an eye exam. In particular embodiments, a first subset of the plurality of vision plan data repositories is associated with a first plan provider; a second subset of the plurality of vision plan data repositories is associated with a second plan provider; and the first plan provider is distinct from the second plan provider. In particular embodiments, the computer-readable instructions further comprise instructions for: determining a benefit value for the first eyewear product option based at least in part on the vision plan data structure; determining a participant cost for the first eyewear product option based at least in part on the vision plan data structure; determining a remaining benefit value based at least in part on the total available benefit value and the benefit value for the first eyewear product option; and updating the graphical user interface to: display the first eyewear product option; display an indication of the benefit value for the first eyewear product option; display an indication of the participant cost for the first eyewear product option; and display an indication of the remaining benefit value. In particular embodiments, the total available benefit value is a total benefit value for a particular component of an eyewear product; and the particular component of the eyewear product is selected from a group consisting of: lenses; frames; and contact lenses. In particular embodiments, the computer-readable instructions further comprise instructions for updating the graphical user interface to an indication of a purchase transaction status based at least in part on the updated transaction status field in the transaction data structure.

### BRIEF DESCRIPTION OF THE DRAWINGS

Various embodiments of the invention will be described below. In the course of the description, reference will be made to the accompanying drawings, which are not necessarily drawn to scale, and wherein:

FIG. 1 is a schematic diagram of a Data Processing System for Calculating Cost and Benefit Parameters according to various embodiments.

FIG. 2 is a schematic diagram of an exemplary computer that may be used in various embodiments, such as in one or more of the devices described in regard to the Data Processing System for Calculating Cost and Benefit Parameters shown in FIG. 1.

FIG. 3 is a representative diagram of an exemplary Eye Care Transaction Data Structure according to various embodiments.

FIG. 4 is a flow chart showing an example of a process for performed by the Dynamic Eye Care Coverage and Benefits Calculation Module.

FIG. 5 is an example of a graphical user interface (GUI) that may be generated by a Data Processing System for Calculating Cost and Benefit Parameters according to various embodiments.

FIG. 6 is another example of a GUI that may be generated by a Data Processing System for Calculating Cost and Benefit Parameters according to various embodiments.

FIG. 7 is another example of a GUI that may be generated by a Data Processing System for Calculating Cost and Benefit Parameters according to various embodiments.

FIG. 8 is another example of a GUI that may be generated by a Data Processing System for Calculating Cost and Benefit Parameters according to various embodiments.

FIG. 9 is another example of a GUI that may be generated by a Data Processing System for Calculating Cost and Benefit Parameters according to various embodiments.

FIG. 10 is another example of a GUI that may be generated by a Data Processing System for Calculating Cost and Benefit Parameters according to various embodiments.

### DETAILED DESCRIPTION

Various embodiments will now be described in greater detail. It should be understood that the invention may be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

### Overview

Many people have eye care insurance plans or other plans that provide eye care and/or optical benefits. Such plans may be obtained through an employer or purchased individually. Typical eye care plans provide benefits that assist the participants in such plans in paying for eye care services and corrective lenses (e.g., glasses, contact lenses). Often the providers of such plans will have agreements with eye care service providers and eyewear manufacturers and distributors to reduce the costs to the providers of particular eye care services and products, thus allowing the providers to obtain vision-related goods and services on behalf of the plan participants at a lower cost to the customers and the providers. The cost of various eye care services and corrective lens components (e.g., frames, lenses, lens coatings) may vary greatly, as may the agreed-upon discounts (if any) for such services and components available with any particular eye care plan.

Because there are so many options available for eye care plans, eye care services, and, especially, optical components and eyewear components, the final cost to a plan participant can be very difficult for the participant to determine during the process of selecting vision-related products and/or services. For example, a participant's plan may include a base amount of benefit coverage for each component of eyewear, and then may cover a percentage of additional components or options that the participant may purchase. In a particular example, the participant's plan may allow the participant to select any frames up to a value of $150 at no cost to the participant, and may also cover 25% of the retail cost of frames above $150 up to $250, and pay no part of the retail cost of frames above $250. In this case, the participant receives essentially “free” frames if the frames have a retail cost at or below $150, pays up to $25 for frames that cost between $150 and $250, and pays $25 plus the full retail cost above $250 for frames that cost more than $250 (e.g., if the frames cost $350, the participant would have to pay $125). There may also be particular coverage for particular brands and types of eyewear components, for example, based on agreements that the plan provider may have in place with particular manufacturers or providers of such components. There may also be varying costs to the participant and/or the plan provider based on the eyewear manufacturer (e.g., based on whether the eyewear manufacturer is affiliated with the relevant insurance provider. The options available to the participant may be dependent, for example, on other options selected by the participant. For example, particular types of lenses may not physically fit into certain frames. There are many other variables that may be factors in determining the options available, for example, for various eyewear components and the ultimate cost to the participant for a particular eyewear component configuration.

In various embodiments, a system for calculating cost and benefit parameters may collect the data associated with a particular plan and a particular plan participant from one or more various sources to determine the specific options available to the particular plan participant under that particular plan. The system may generate a data structure to track the participant's selections during the eye care product and/or services transaction, while maintaining data indicating the various options that are available to the participant based on the particular plan and the selections made thus far. The system may calculate the cost to the particular participant, as well as the available and committed benefits to the participant under the particular plan. The system may then dynamically update the costs and benefits as the participant makes further selections and proceeds through the process of purchasing vision-related products and/or services. By providing this dynamically updated cost and benefit information to the participant during the purchasing process, the system may greatly enhance the participant's knowledge of the available plan benefits and prospective cost of the vision-related products and/or services, allowing the participant to make a more informed eye care purchasing decision. By using the information generated and presented by the disclosed systems, the participant can choose to maximize the usage of their plan benefits and/or ensure that the total final cost of the vision-related products and/or services remains within the participant's budget. The system may also be beneficial in that it helps the insured better understand the value of their vision coverage.

### Exemplary Technical Platforms

As will be appreciated by one skilled in the relevant field, the present systems may be, for example, embodied as a computer system, a method, or a computer program product. Accordingly, various embodiments may take the form of an entirely hardware embodiment, an entirely software embodiment, or an embodiment combining software and hardware aspects. Furthermore, particular embodiments may take the form of a computer program product stored on a computer-readable storage medium having computer-readable instructions (e.g., software) embodied in the storage medium. Various embodiments may take the form of web-implemented computer software. Any suitable computer-readable storage medium may be utilized including, for example, hard disks, compact disks, DVDs, optical storage devices, and/or magnetic storage devices.

Various embodiments are described below with reference to block diagrams and flowchart illustrations of methods, apparatuses (e.g., devices, systems), and computer program products. It should be understood that each block of the block diagrams and flowchart illustrations, and combinations of blocks in the block diagrams and flowchart illustrations, respectively, can be implemented by a computer executing computer program instructions. These computer program instructions may be loaded onto a general purpose computer, special purpose computer, or other programmable data processing apparatus to produce a machine, such that the instructions which execute on the computer or other programmable data processing apparatus to create means for implementing the functions specified in the flowchart block or blocks.

These computer program instructions may also be stored in a computer-readable memory that can direct a computer or other programmable data processing apparatus to function in a particular manner such that the instructions stored in the computer-readable memory produce an article of manufacture that is configured for implementing the function specified in the flowchart block or blocks. The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide steps for implementing the functions specified in the flowchart block or blocks.

Accordingly, blocks of the block diagrams and flowchart illustrations support combinations of mechanisms for performing the specified functions, combinations of steps for performing the specified functions, and program instructions for performing the specified functions. It should also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and other hardware executing appropriate computer instructions.

### Exemplary System Architecture

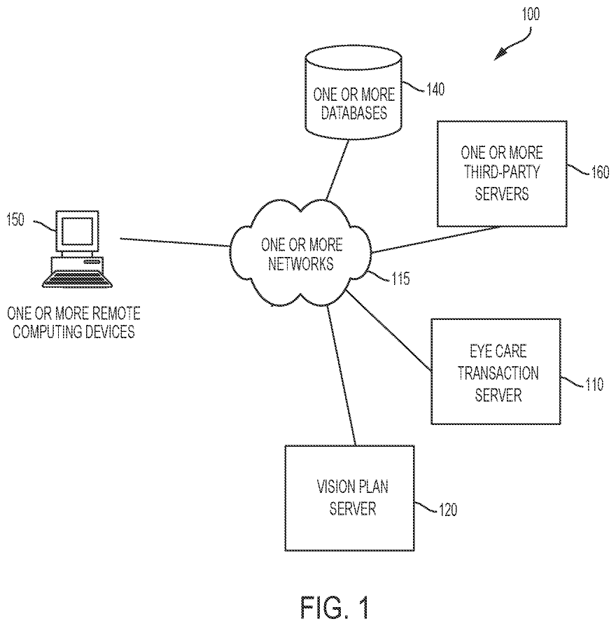

FIG. 1 is a block diagram of a Data Processing System for Calculating Cost and Benefit Parameters 100 according to a particular embodiment. As may be understood from this figure, the Data Processing System for Calculating Cost and Benefit Parameters 100 may include an Eye Care Transaction Server 110, a Vision Plan Server 120, one or more third-party servers 160, one or more networks 115, one or more remote computing devices 130 (e.g., such as a smart phone, a tablet computer, a wearable computing device, a laptop computer, a desktop computer, an eye care provider computer, a point-of-sale (POS) device, etc.), and one or more databases 140 (e.g., cloud storage). In particular embodiments, the one or more networks 115 facilitate communication between the one or more remote computing devices 150, the Eye Care Transaction Server 110, the Vision Plan Server 120, and/or the one or more databases 140. Any of the one or more databases 140 may be a data repository that captures and/or stores data that may be used to determine eye care plan benefits, costs, discounts, eyewear components, other vision-related products, eye care services, etc. In addition, or instead, any of the one or more databases 140 may be a data repository configured to capture, store, and/or determine criteria that may be used to determine eye care and/or vision plan data. The Vision Plan Server 120 and/or the one or more third-party servers 160 may each be one or more servers that store and/or provide vision plan data associated with one or more participants in one or more vision plans.

The Vision Plan Server 120 may collect and store vision plan data from one or more of various vision plan provider systems (e.g., the one or more third-party servers 160). The Vision Plan Server 120 may organize such data using one or more suitable data structures so that such data may be readily utilized by, for example, the Eye Care Transaction Server 110 to perform data processing for calculating cost and benefit parameters as described herein. In various embodiments, the Vision Plan Server 120 may obtain such plan data using data mining techniques. In various embodiments, the Vision Plan Server 120 may also, or instead, obtain such plan data in real-time, for example in response to a query from the Eye Care Transaction Server 110, to ensure that the Eye Care Transaction Server 110 uses the most up-to-date information in processing a transaction for a particular participant in a particular plan.

The one or more networks 115 may include any of a variety of types of wired or wireless computer networks, or any combination thereof, such as the Internet, a private intranet, a mesh network, a public switch telephone network (PSTN), or any other type of network (e.g., a network that uses Bluetooth or near field communications to facilitate communication between computing devices). The communication link between any of the components of the System 100, such as the one or more remote computing devices 150 and the Eye Care Transaction Server 110 may be, for example, implemented via a Local Area Network (LAN) or via the Internet.

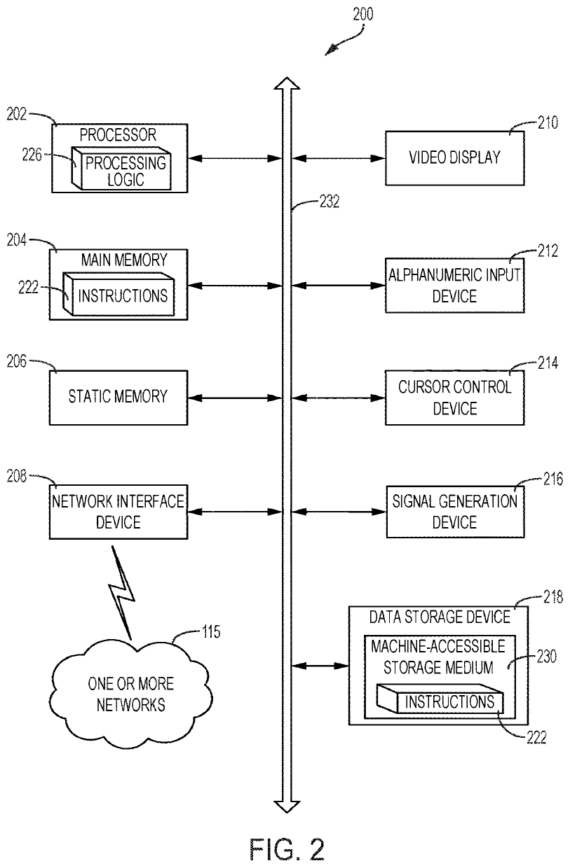

FIG. 2 illustrates a diagrammatic representation of a computer architecture 200 that can be used within the Data Processing System for Calculating Cost and Benefit Parameters 100, for example, as a client computer (e.g., one of the one or more remote computing devices 150 shown in FIG. 1), or as a server computer (e.g., the Eye Care Transaction Server 110 shown in FIG. 1).

In particular embodiments, the computing device 200 may be connected (e.g., networked) to other computing devices via a LAN, an intranet, an extranet, and/or the Internet. As noted above, the computing device 200 may operate in the capacity of a server and/or a client computing device in a client-server network environment, and/or as a peer computing device in a peer-to-peer (or distributed) network environment. The computing device 200 may be a desktop personal computing device (PC), a tablet PC, a set-top box (STB), a Personal Digital Assistant (PDA), a cellular telephone, a web appliance, a server, a network router, a switch or bridge, or any other computing device capable of executing a set of instructions (sequential or otherwise) that specify actions to be taken by that computing device. Further, while only a single computing device is illustrated, the term “computing device” shall also be taken to include any collection of computing devices that individually or jointly execute a set (or multiple sets) of instructions to perform any one or more of the methodologies discussed herein.

Returning to FIG. 2, an exemplary computing device 200 includes a processing device 202, a main memory 204 (e.g., read-only memory (ROM), flash memory, dynamic random access memory (DRAM) such as synchronous DRAM (SDRAM) or Rambus DRAM (RDRAM), etc.), a static memory 206 (e.g., flash memory, static random access memory (SRAM), etc.), and a data storage device 218, which communicate with each other via a bus 232.

The processing device 202 represents one or more general-purpose or specific processing devices such as a microprocessor, a central processing unit, or the like. More particularly, the processing device 202 may be a complex instruction set computing (CISC) microprocessor, reduced instruction set computing (RISC) microprocessor, very long instruction word (VLIW) microprocessor, or processor implementing other instruction sets, or processors implementing a combination of instruction sets. The processing device 202 may also be one or more special-purpose processing devices such as an application specific integrated circuit (ASIC), a field programmable gate array (FPGA), a digital signal processor (DSP), network processor, or the like. The processing device 202 may be configured to execute processing logic 226 for performing various operations and steps discussed herein.

The computing device 200 may further include a network interface device 208. The computing device 200 also may include a video display unit 210 (e.g., a liquid crystal display (LCD), a light-emitting diode (LED) display, an organic LED (OLED) display, a cathode ray tube (CRT), etc.), an alphanumeric input device 212 (e.g., a keyboard), a cursor control device 214 (e.g., a mouse), and a signal generation device 216 (e.g., a speaker).

The data storage device 218 may include a non-transitory computing device-accessible storage medium 230 (also known as a non-transitory computing device-readable storage medium or a non-transitory computer-readable medium) on which may be stored one or more sets of instructions (e.g., software 222) embodying any one or more of the methodologies or functions described herein. The software 222 may also reside, completely or at least partially, within the main memory 204 and/or within the processing device 202 during execution thereof by the computing device 200—the main memory 204 and the processing device 202 also constituting computing device-accessible storage media. The software 222 may further be transmitted or received over a network 115 via a network interface device 208.

While the computing device-accessible storage medium 230 is shown in an exemplary embodiment to be a single medium, the terms “computing device-accessible storage medium” and “computer-readable medium” should be understood to include a single medium or multiple media (e.g., a centralized or distributed database, and/or associated caches and servers) that store the one or more sets of instructions. The terms “computing device-accessible storage medium” and “computer-readable medium” should also be understood to include any device that is capable of storing, encoding or carrying a set of instructions for execution by the computing device and that cause the computing device to perform any one or more of the methodologies of the present invention. The terms “computing device-accessible storage medium” and “computer-readable medium” should accordingly be understood to include, but not be limited to, solid-state memories, optical and magnetic media, etc.

### “Big Data” Processing and Neural Networks for Calculating Cost and Benefit Parameters

In various embodiments, and as described in further detail below, the system may use “big data” techniques (e.g., machine learning techniques that, for example, use artificial intelligence to identify one or more patterns within a large set of data) to process vision plan data and participant data that may have been previously stored to determine one or more options available to a participant and to determine participant costs and benefits. The system may also use such techniques to determine one or more underutilized options and/or other friction points in the vision-related product and/or services purchasing process. In various embodiments, the system may use big data processing techniques to mine the purchase process data previously stored for multiple past purchases of vision-related products and/or services to determine whether to adjust the various options and offers made to participants to increase the effectiveness (e.g., in terms of reducing provider costs, increasing participant benefits, increasing provider profit, etc.) of the purchase process. For example, in various embodiments, the system may use machine learning to analyze a large body of data regarding past purchases and the marketing associated with those past purchases to optimize one or more marketing and/or customer-education activities in a way that is projected to improve and/or optimize customer purchasing behavior and/or that is projected to improve and/or optimize a customer's usage of their vision plan benefits. In various embodiments, the system may use big data processing techniques to perform any of the computer-implemented processes (e.g., execution of any of the modules) described herein to determine any of the plan options available to a participant and/or the costs, benefits (allocated and/or remaining), savings, marketing, customer education etc. associated with a particular process of purchasing vision-related products and/or services.

In particular embodiments, one or more neural networks may be used to implement any of the big data processing techniques described herein. A neural network, according to various embodiments, comprises: (1) a plurality of nodes that mimic the operation of the human brain; (2) a training mechanism that analyzes supplied information, and (3) an eye care transaction engine for determining one or more vision plans associated with a particular participant, determining costs, benefits, and/or savings associated with a particular participant and plan, determining options at a particular point in the process of purchasing vision-related products and/or services based on past option selections and/or the associated plan, determining status and data associated with an interaction with a particular participant, and/or determining one or more friction points in a purchasing process and associated adjustments the options and/or offers made to participants to increase the effectiveness of that purchase process or otherwise reduce the friction at such identified one or more friction points. The neural network may also perform any of the processing (e.g., execute any of the modules) described herein to determine options, costs, benefits, savings, etc. as described herein. In various embodiments, each of the nodes comprises one or more weighted input connections, at least one transfer function that combines the inputs, and an output connection. In particular embodiments, the neural network is a variational autoencoder (AE) neural network, a denoising AE neural network, or any other suitable neural network.

In various embodiments, for example, where the system employs a neural network, the system may infer information (e.g., regarding preferred options, preferences, friction points, etc.) based on known and/or available data (e.g., plan data, participant data, selection data, etc.). In particular embodiments, the system may use a neural network and integrate inference into its training mechanism to infer information that may then be used in reducing friction in the purchase process and/or in determining options, costs, savings, preferences, etc.

In various embodiments, the system may use predictive analytics to determine preferred options, preferences, friction points, etc. For example, the system may utilize one or more of a variety of statistical techniques in conjunction with “big data” processing, including as described herein. In particular embodiments, the system may employ any one or more of a variety of statistical techniques in conjunction with data mining, predictive modeling, and/or machine learning (e.g., as described herein) to analyze current participant information and/or historical participant information to generate predictions regarding preferences and preferred or more appealing options as described herein. In particular embodiments, the system may employ machine learning in implementing the disclosed systems and methods to better determine appealing participant options and/or reduce friction and any of various points in the process of purchasing vision-related products and/or services.

### Eye Care Transaction System Overview

To facilitate the purchase of eye care and eyewear, the system may gather information on various vision plans, for example, that may be accepted by the owner or operator of the disclosed eye care transaction system. Referring again to FIG. 1, the Vision Plan Server 120 may query and/or receive vision plan data from the One or More Third-Party Servers 160 and store such data for use in eye care transactions and/or interactions. In various embodiments, the Vision Plan Server 120 may perform data mining operations across multiple devices (represented as the One or More Third-Party Servers 160) that may store vision plan data. The Vision Plan Server 120 may organize the collected vision plan data into one or more data structures, each representing a particular vision plan and including data representing one or more aspects of the respective particular vision plan (e.g., benefits, options, costs, participating eyewear component vendors, participating eye care providers, plan identifying information, etc.). The Vision Plan Server 120 may create one or more templates for respective transaction and/or interaction data structures used in the purchase transactions and/or participant interactions based on these one or more data structures as described in more detail below.

When a particular participant in a vision plan visits an eye care provider, or a website of an eye care provider, the Eye Care Transaction Server 110 may generate and present an interface to collect information from a user that the system can then use to determine if the user is a participant in an eye care plan. The system may collect identifying information from the user (e.g., name, phone number, employee number, employer, social security number, address, etc.) and then use that information to determine whether the user is an active plan participant, for example, by querying a server that may identify plan participants (e.g., the Vision Plan Server 120, the One or More Third-Party Servers 160). Instead, or in addition, in various embodiments, the interface generated and presented by the Eye Care Transaction Server 110 may collect information about a vision plan in which the user is a participant, such as the relevant plan name, plan number, group number, etc. The system may use this information to confirm that the user is an active plan participant, for example, by querying a server that may be configured to identify such plan participants (e.g., the Vision Plan Server 120, the One or More Third-Party Servers 160).

In response to determining that the user is a plan participant in a particular vision plan, the Eye Care Transaction Server 110 may determine the various parameters associated with that particular plan and user, for example by querying the Vision Plan Server 120. Such parameters may include the services available (e.g., eye exam), the products available (e.g., glasses, contact lenses), and the various options associated with both the plan and the user (e.g., particular types of tests available with the eye exam, particular lens types available, particular frame types available, etc.). Such parameters may also, or instead, include respective costs and/or coverage associated with any such services, products, and related options. The Eye Care Transaction Server 110 may generate a data structure for the particular transaction/interaction initiated by this particular user to store such information. The Eye Care Transaction Server 110 may use a template of a transaction or interaction data structure received from the Vision Plan Server 120 or may generate the data structure using plan data received from the Vision Plan Server 120. The system may update this data structure as needed during the transaction/interaction to maintain a record of the selections of the participant during the eye care purchase transaction process, the position of the participant within the eye care purchase transaction process, and/or any other suitable data.

In various embodiments, the Eye Care Transaction Server 110 may generate an interface indicating to the participant the particular eye care options available to the participant at that point in the eye care purchase interaction and transaction process. The interface may also indicate the cost, coverage, and/or benefit parameters associated with the particular options. The Eye Care Transaction Server 110 may then collect participant selections of particular options via such an interface at the various points in the purchase transaction process where participant input is needed. The system may then dynamically update the options, cost, coverage, and/or benefit parameters available based on the participant's selections and present the participant with a current and running account of the benefits and costs that the participant has selected and has available based on the selections made thus far. After the participant has completed a set of selections that can be used to finalize a purchase of one or more particular products and/or services, the Eye Care Transaction Server 110 may allow the participant to finalize the purchase and/or begin the purchase process for one or more subsequent products or services.

### Eye Care Transaction Data Structure

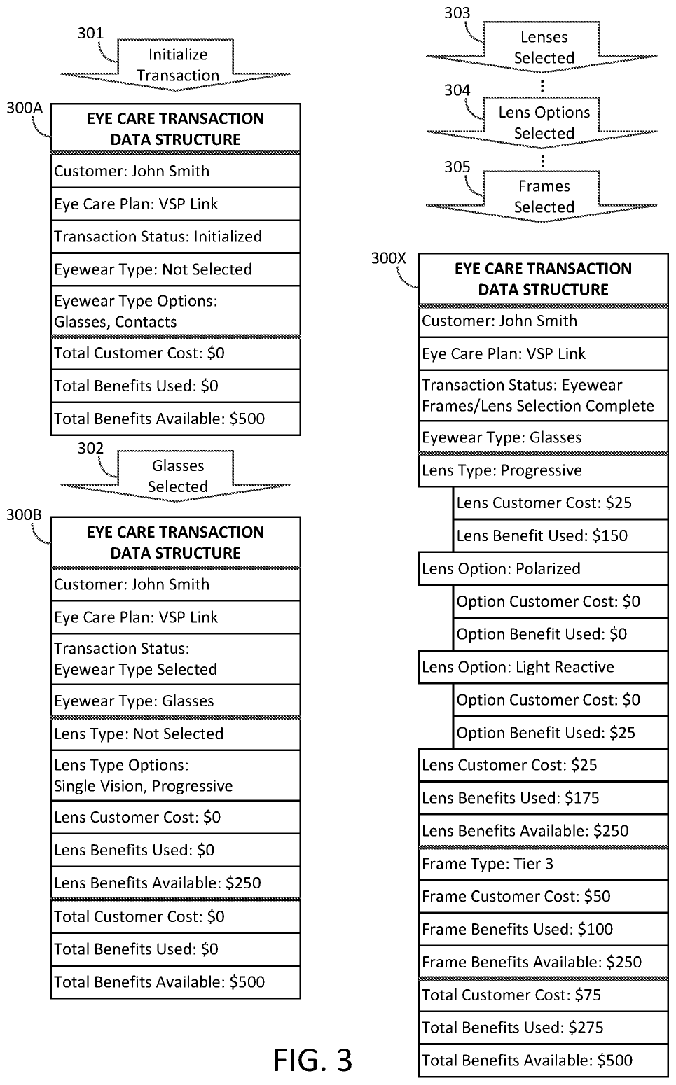

In various embodiments, the system may use one or more data structures for each eye care transaction and/or interaction with a participant. FIG. 3 illustrates an exemplary Eye Care Transaction Data Structure 300 representing a data structure that may be generated, stored, modified, and/or supplemented during a purchase process and/or participant interaction. In response to receiving an indication 301 to initialize an eye care transaction, the system may collect participant information and obtain related plan information using the collected participant information. The system may then generate the initial Data Structure 300A using the initial information received and/or determined for the eye care transaction. For example, as seen in FIG. 3, initial Data Structure 300A may include the customer name (“John Smith”) and plan identifier (“VSP Link”) as provided or selected by the participant (e.g., via a graphical user interface (GUI)). The initial Data Structure 300A may also include plan information that the system determined based on information provided by the participant, such as total benefits available (“$500”) and eyewear type options (“glasses, contacts”). As the data structure has just been initialized, the initial Data Structure 300A may also contain default information in various fields (e.g., total cost and total benefits used set to “$0,” type of eyewear set to “not selected,” etc.). A status associated with this transaction or interaction may be indicated as “initialized” in the initial Data Structure 300A. The information in the initial Data Structure 300A may be used to generate an interface (e.g., a GUI) that may be presented to the user to facilitate the vision-related products and/or services purchase process, as described herein.

Note that the transaction/interaction status field may be particularly useful in the event that a transaction is interrupted before completion. In various embodiments, the transaction data structure may be stored for later use if a transaction times out or is otherwise left incomplete. In such cases, the system may update the data structure to indicate that the transaction has timed out. If the system detects the participant interacting with the system at a future time, the system may offer to continue the transaction with the participant. The system can determine the point in the purchase process that the transaction left off based on the transaction status stored in the data structure. The system can then resume the transaction with the participant at that point, thus avoiding having to ask the participant to repeat the selections made in the participant's first effort to complete the purchase transaction.

In response to receiving an indication 302 that an available option (e.g., optical option) has been selected by the participant (in this example, the participant selected “glasses” as the eyewear type), the system may update the initial Data Structure 300A to the updated Data Structure 300B using the selection information. That is, the system may update and expand the data structure based on the selection. For example, as seen in FIG. 3, the updated Data Structure 300B may include an updated eyewear type field, updated to indicate “glasses.” Where applicable, the total customer cost and savings may also be updated based on the user selection (not applicable yet in this example). The updated Data Structure 300B may also be supplemented to include one or more options and/or benefits available based on the participant's selections, such as one or more lens options (e.g., lens type “not selected,” lens options “single vision, progressive,” etc.) and one or more lens benefits available (e.g., “$250”). The updated Data Structure 300B may also be supplemented to include savings, cost, and available benefits information based on the participant's selections (in the example of FIG. 3, since no particular options are selected yet, the lens customer cost is “$0” and the lens benefits used is “$0”).

The system may update, modify, supplement, and/or reduce the Data Structure 300 as often as necessary in response to one or more actions that may occur in the purchase process. For example, following each of the participant selections of lenses 303, lens options 304, and frames 305, as well as any other selections, the system may update and/or modify the Data Structure 300 as needed based on the selections and/or actions. The system may use each updated version of the data structure to present information to the participant as described herein.

After the participant has completed making selections of the available options, the final Data Structure 300X may be updated and modified using the participant's selections and the plan data. In the example represented by the final Data Structure 300X, the participant has selected progressive lenses with the options of polarization and light reactivity. As can be seen in the example, the progressive lenses used $150 of the participant's lens benefits and will cost the participant $25. The option of polarization is offered at no cost by the provider, and so costs the participant nothing and does not reduce the participant's lens benefit. The option of light reactivity used $25 of the participant's lens benefits and will cost the participant $0. Thus, these lenses and options consume $175 of the participant's $250 available lens benefit (thus leaving the participant with $75 of remaining available lens benefit) and will cost the participant $50.

Further in this example, the participant has selected “Tier 3” frames which will cost the participant $50 and use $100 of the participant's frame benefits (thus leaving the participant with $150 of remaining available frame benefit).

Because of these selections, the participant will incur a total cost of $75 and consume $275 of the participant's eyewear benefit, leaving $225 of the participant's eyewear benefit ($75 in lens benefits and $150 in frame benefits) available. Note that in some embodiments, the remaining benefit values may also be calculated and stored in the Data Structure 300, while in other embodiments, these values may be calculated “on the fly” as needed based on the total benefit values and used benefit values. The system may present the benefit values and any other information from the Data Structure 300 at each or any point during the purchase process to assist the participant in making educated decisions regarding the selection of options for the participant's eye care.

### Dynamic Eye Care Coverage and Benefits Calculation Module

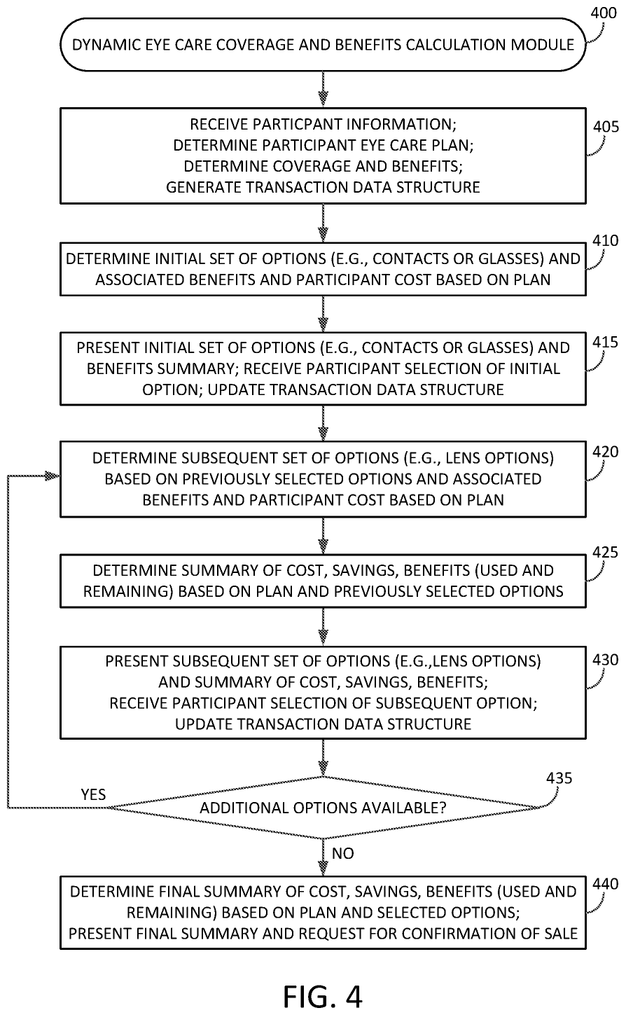

FIG. 4 shows an example process performed by the Dynamic Eye Care Coverage and Benefits Calculation Module 400. The system may execute this module in response to the initiation of an eye care purchase process, for example as described herein. At Step 405, the system may receive initial information that it may use to perform initialization activities for a purchase transaction. For example, the system may receive participant information such as the participant's name, social security number, employee identification number, and/or other identifying information (e.g., via a GUI). The system may also receive information identifying the vision plan in which the participant participates, or the system may determine the plan based on the participant identifying information. The system may use the received information to determine the particular plan (if necessary) and plan information. For example, the system may query a vision plan server (e.g., the vision Plan Server 120) for particular plan information for the plan in which the participant participates. The system may use the plan information to determine the particular benefits and coverage associated with the particular plan and the particular participant. The system may also, at Step 405, generate a transaction data structure to store information regarding the participant's eye care purchase transaction and track the participant's selections and progress in the transaction process.

At Step 410, the system may determine the initial set of options (e.g., optical options, eyewear options, eye care options, etc.) to present to the participant in the purchase process. For example, the system may determine that the participant will need to select a type of eyewear (e.g., glasses or contact lenses) before making other selections. The system may then determine the particular benefits and costs associated with each of the eyewear type options.

At Step 415, the system may present the initial set of options to the participant, for example, in a GUI. The form of presentation of this set of options may also include a selectable control associated with each option allowing the participant to select one of more of the options, as described in more detail below. Included with the options, the system may present a summary or other indication of the benefits available to the participant based on the participant's plan. Further, at Step 415, the system may detect the participant selection of a particular option from among the set of options (e.g., a selection of glasses from the options of glasses or contact lenses). Responsive to this selection, the system may update the transaction data structure to indicate an updated transaction status and the selected option.

At Step 420, based on the selected particular option (e.g., glasses), the system may determine a subsequent set of options (e.g., available lens types) that may be associated with the selected particular option and the associated benefits and costs for each of the further options. In various embodiments, some options may have particular subsequent options associated with them that are not associated with other options. For example, particular types of lenses may not fit in every type of frame, so in response to a participant selecting a particular type of lens, the system may present a listing of only the particular types of frames that are capable of being fitted with the selected particular type of lenses. The system may also determine the participant cost and available benefit for those particular types of frames. In various embodiments, the system may supplement the transaction data structure with this information.

At Step 425, the system may determine and/or update the total availability and usage of benefits associated with the participant's plan based on all the options selected thus far. In various embodiments, the system may supplement the transaction data structure with this information.

At Step 430, the system may present the subsequent set of options to the participant, for example, in a GUI. Here again, the form of presentation this set of options may also include a selectable control associated with each option allowing the participant to select one of more of the options, as described in regard to the user interfaces described below. Included with the options, the system may present a summary or other indication of the benefits available to the participant based on the participant's plan. The system may also present to the participant a summary or other indication of the total availability and usage of benefits associated with the participant's plan based on all the options selected thus far. Also, at Step 430, the system may detect the participant's selection of a particular option from among the set of subsequent options (e.g., a selection of progressive or single vision from the available lens types). Responsive to this selection, the system may update the transaction data structure to indicate an updated transaction status and the selected subsequent option.

At Step 435, the system may determine whether there are other possible options to present to the participant. For example, where the participant has selected lenses, frames, and lens options for a pair of glasses, there may be no other options remaining and the purchase may be in condition for completion. In such cases, the system may, at Step 440, determine a final indication or summary of the costs, savings, and benefits used for the participant and present this information with indications of the selected products and/or services and a user-selectable control allowing the participant to complete the purchase. The system may also update the transaction data structure with the final product selections, the final costs, savings, and benefits information, and to indicate that the purchase process is at a purchase confirmation status.

If, at Step 435, the system determines that there are additional options that the participant will need to select, the system returns to Step 420 to present subsequent options to the participant. For example, the participant may not yet have selected lens options, or frames to go with selected lenses, etc., and therefore the system may proceed to the next appropriate step in the purchasing process.

### Exemplary User Experience

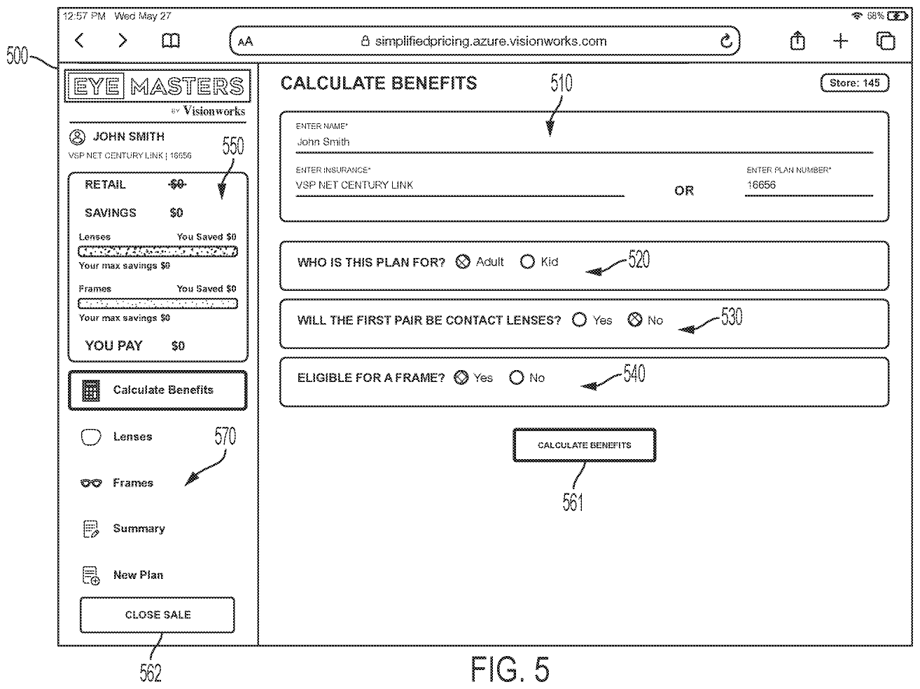

In various embodiments, the system may collect information from a participant and present information representing the processing and analysis performed by the system using one or more user interfaces, such as GUIs. FIG. 5 illustrates an example interface 500 that the system (e.g., the Eye Care Transaction Server 110) may present to a participant at the initiation of a purchase transaction process for eye care products and/or services. The interface 500 may include one or more fields in which a user may enter information that the system can then use to determine plan information for the particular user, including whether the user is an active participant in a vision plan. For example, the interface 500 may include the user information fields 510 that allow the user to provide identifying information, such as one or more of the user's name, phone number, employee number, employer, social security number, address, etc. In various embodiments, the system may use this information to determine the plan associated with the user (if any). Alternatively, or in addition, the user information fields 510 may allow the user to provide identifying information for a vision plan in which the user is a participant, such as one or more of a plan name, a plan number, a group number, etc. The system may use this information to confirm that the user is an active plan participant, for example, by querying a server that may identify plan participants (e.g., the Vision Plan Server 120, the One or More Third-Party Servers 160).

To further facilitate the purchase process, the interface 500 may include one or more fields that allow the system to determine the available and/or preferred options to present to the participant during the purchase process. For example, the interface 500 may include the demographic fields 520 that may allow the participant to indicate, for example, whether the services and/or products requested are for an adult or a child. The interface 500 may also, or instead, include a product type field 530 that may allow the participant to indicate a desired product or service type, for example, glasses or contact lenses. The interface 500 may also, or instead, include an eligibility field 540 that may allow the participant to indicate an eligibility for particular products or services, for example, whether the participant is eligible for a set of frames.

The interface 500 may further present plan information to the participant. For example, the plan information section 550 may include a summary of the benefits available to the participant, the benefits used, and the cost to the participant. The system may determine this information based on the plan data and the particular products, services, and/or options selected by the participant at this point in the purchase process. The interface 500 may also include an indicator of the transaction status, such as the status indicator 570 that highlights the particular current position of the participant in the purchase process while also showing (but not highlighting) one or more other positions in the purchase process.

The interface 500 may include one or more user-selectable controls, such as the calculate benefits control 561 that allows the user to request updated benefits information, for example after entering information in one or more of the fields of interface 500. The interface may also include a purchase control 562 that may allow the participant to complete the purchase and provide payment or otherwise terminate the purchase process.

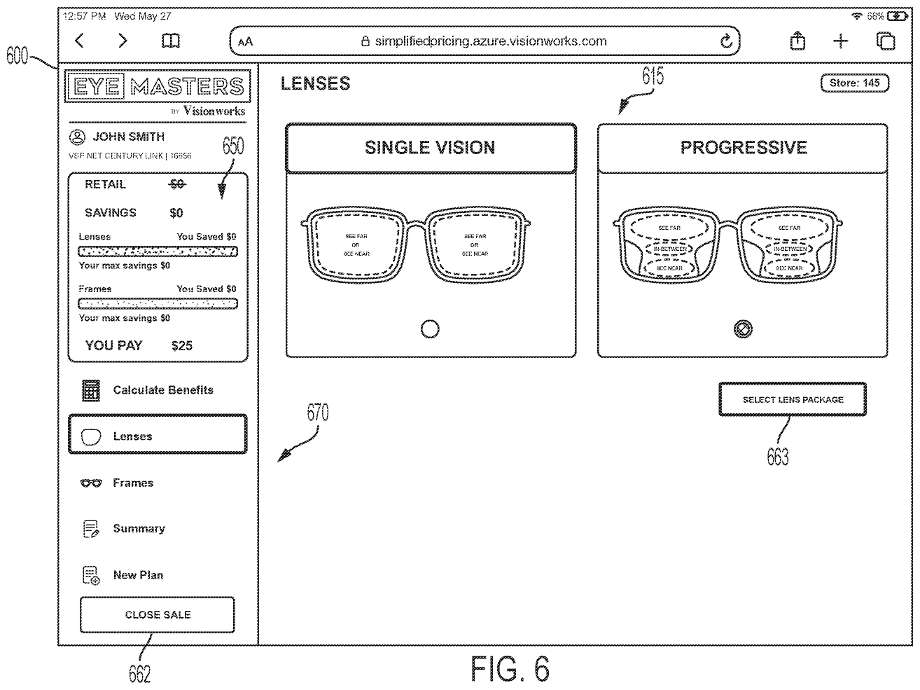

FIG. 6 illustrates an example interface 600 that the system (e.g., the Eye Care Transaction Server 110) may present to a participant during the purchase transaction process for eye care products and/or services. After receiving one or more selections and/or information from the participant (e.g., at an interface such as the interface 500 in FIG. 5, where the participant selected glasses as the desired eye care product), the system may generate the interface 600 to request and receive participant selections of particular options for a particular component of eyewear. The system may present the options in the selection section 615, which in FIG. 6 offers the participant the opportunity to select either single vision or progressive lenses for a pair of glasses being purchased by the participant in this particular example purchase transaction. The interface 600 may include an indicator of the transaction status, such as the status indicator 670 that highlights the participant at the lenses selection point in the purchase process.

The system may detect the participant's selection of the type of lenses desired and, in response, may present an updated plan information section 650 that includes a summary of the benefits available to the participant, the benefits used, and the cost to the participant based on the participant's selection of a type of lenses. For example, in the exemplary interface shown in FIG. 6, the participant has selected progressive lenses, which, based on the participant's vision plan and as shown in in section 650, will cost the participant $25, but will not reduce the participant's total available lens benefit. The system may proceed to the next point in the purchase process in response to detecting the participant's selection of the control 663, or may close the sale and/or terminate the purchase process in response to detecting the participant's selected of the control 662.

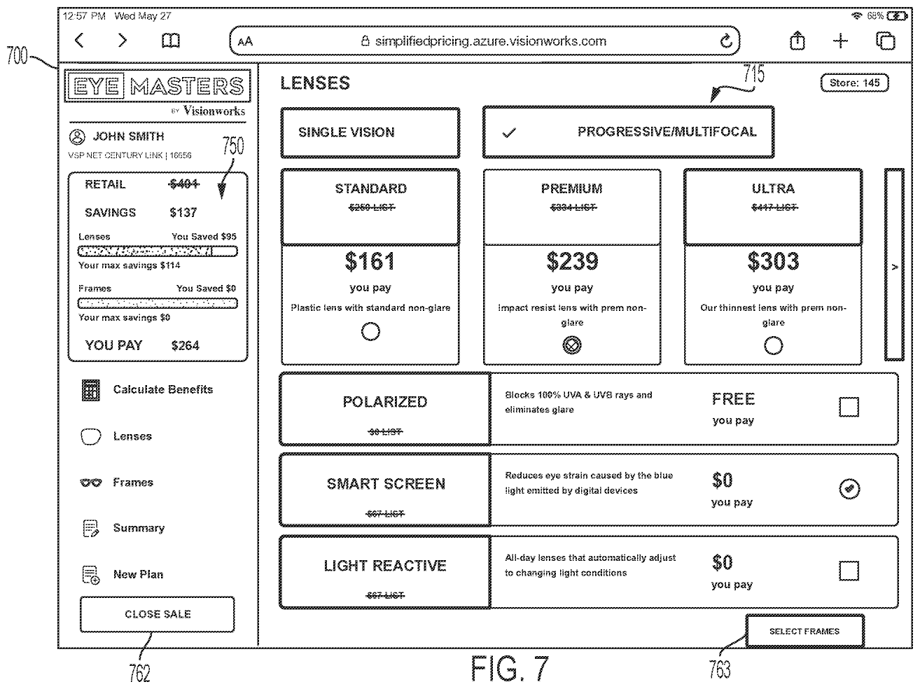

FIG. 7 illustrates another example interface 700 that the system (e.g., the Eye Care Transaction Server 110) may present to a participant during the purchase transaction process for eye care products and/or services. After receiving one or more selections and/or information from the participant (e.g., at an interface such as the interface 600 in FIG. 6, where the participant selected progressive lenses as the desired lens type), the system may generate the interface 700 to request and receive participant selections of additional options for a particular component of eyewear. The system may present the available options in the selection section 715, which in FIG. 7 offers the participant the opportunity to select a quality of lens (e.g., “standard,” “premium,” or “ultra”) and one or more of various lens options (e.g., “polarized,” “smart screen,” “light reactive”).

The system may detect the participant's selection of desired lens options and, in response, may present the updated plan information section 750 that includes a summary of the benefits available to the participant, the benefits used, and the cost to participant based on the participant's selection of lens options. For example, in the exemplary interface shown in FIG. 7, the participant has selected premium grade lenses and the “smart screen” lens option. In this example, based in the participant's vision plan and as shown in section 750, this combination of lenses (participant cost $25) and lens options (participant cost $239) will cost the participant $264 and will incur a cost of $95 against the participant's total available lens benefit of $114. Also, as displayed in the section 750, the participant is saving $95 over the retail cost of the premium lenses ($334). As will be appreciated, providing such updated and running benefit, savings, and cost information during the purchase process may be very helpful in assisting the participant in making informed decisions about the use of the participant's plan benefits. The system may proceed to the next point in the purchase process in response to detecting the participant's selection of the control 763, or may close the sale and/or terminate the purchase process in response to detecting the participant's selection of control 762.

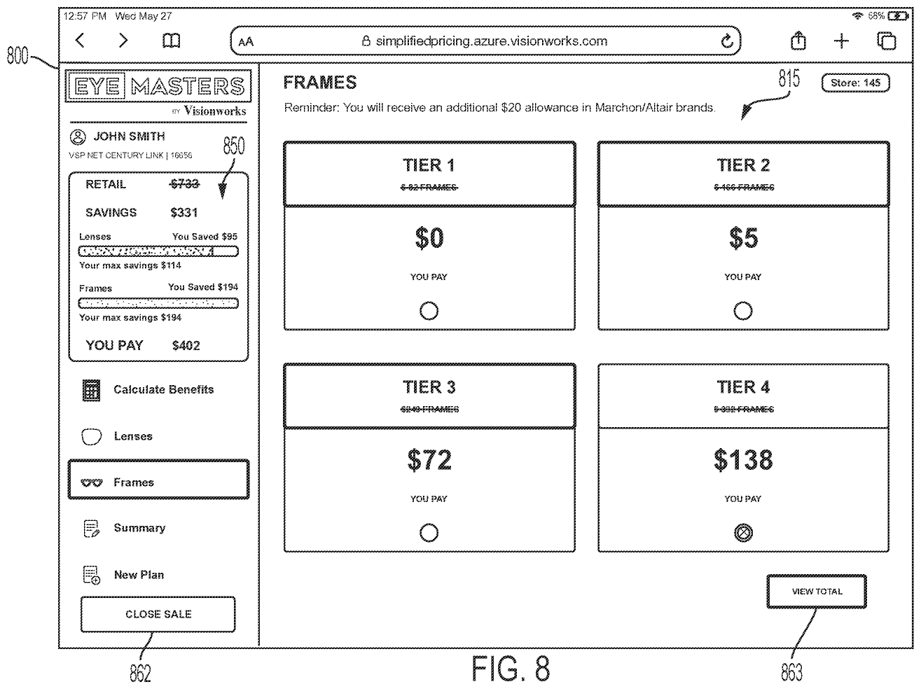

FIG. 8 illustrates another example interface 800 that the system (e.g., via the Eye Care Transaction Server 110) may present to a participant during the purchase transaction process for eye care products and/or services. After receiving one or more selections and/or information from the participant (e.g., at an interface such as the interface 700 in FIG. 7, where the participant selected various lens options for progressive lenses), the system may generate the interface 800 to request and receive participant selections of additional options for a particular component of eyewear (e.g., frames). The system may present the available options in the selection section 815, which in FIG. 8 offers the participant the opportunity to select a tier of frames (e.g., tier 1, 2, 3, or 4). In particular embodiments, frames may be organized into tiers based on cost or other criteria. For example, the tiers may correspond to a range of frames from least expensive frames to most expensive frames. Frame expense may be based on a variety of criteria other than, or including, actual cost. For example, a vision plan provider may have agreements with one or more particular frame manufacturers or distributors that allow the provider to pay less for the associated frames. Frame tiers may also, or instead, be based on the cost to the participant. For example, Tier 1 frames may have a minimal participant cost while higher tier frames may have increasingly higher participant costs.

The system may detect the participant's selection of a desired frame tier and, in response, may present the updated plan information section 850 that includes a summary of the benefits available to the participant, the benefits used, and the cost to participant based on the participant's selection of a frame tier. For example, in the exemplary interface shown in FIG. 8, the participant has selected Tier 4 frames. Continuing with the example used in FIG. 7, based in the participant's vision plan and as shown in in section 850, this combination of frames (participant cost $138), lenses (participant cost $25) and lens options (participant cost $239) will cost the participant $402 and will incur a cost of $194 against the participant's total available frame benefit of $194 and a cost of $95 against the participant's total available lens benefit of $114. Also as displayed in the section 850, the participant is saving $194 over the retail cost of the Tier 4 frames ($332). The system may proceed to the next point in the purchase process in response to detecting the participant's selection of the control 863, or may close the sale and/or terminate the purchase process in response to detecting the participant's selection of the control 862.

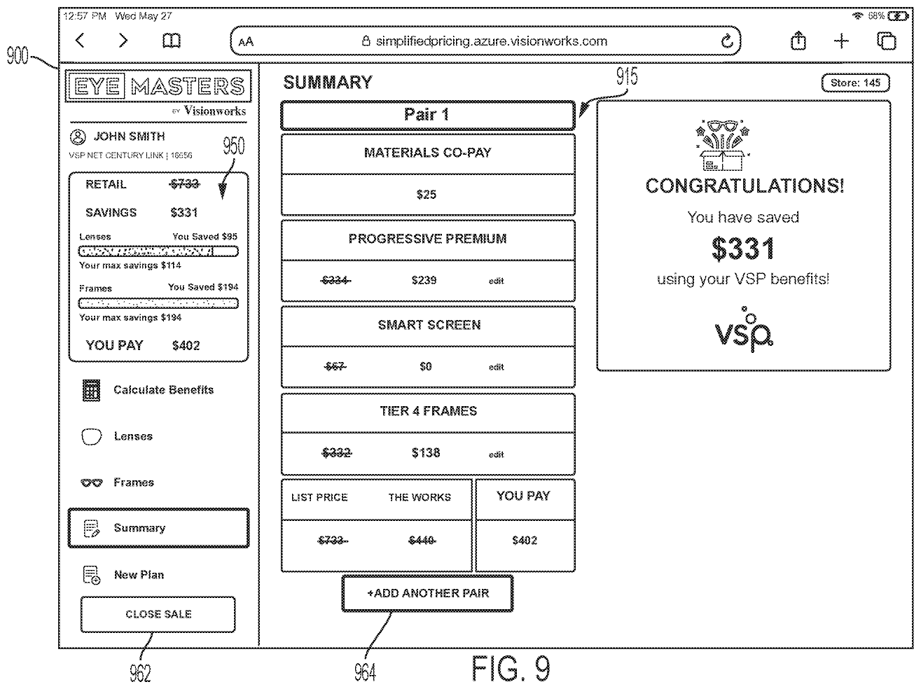

FIG. 9 illustrates another example interface 900 that the system (e.g., via the Eye Care Transaction Server 110) may present to a participant during the purchase transaction process for eye care products and/or services. After receiving one or more selections and/or information from the participant associated with the purchase of a particular eye care product or service (e.g., at the interfaces described in regard to FIGS. 5-8), the system may generate the interface 900 to present a summary of the options selected by the participant for the particular product or service and the total cost, benefit, and savings. The system may present the summary section 915 displaying the selected product or service and associated options, which in FIG. 9 represents one pair of glasses with progressive premium lenses, various lens options, and Tier 4 frames, continuing with the example used in the previously described interfaces. Also shown in the section 915 is the retail cost associated with each item and the respective participant cost, representing the savings the participant achieved using the vision plan.

The system may present the final plan information section 950 that includes a summary of the benefits available to the participant, the total benefits used for the particular product or service, and the total cost to the participant for the particular product or service based on the participant's selections. For example, in the exemplary interface shown in FIG. 9, and continuing with the example used in the previous interfaces, the participant's various selections resulted in a combination of frames (participant cost $138), lenses (participant cost $25) and lens options (participant cost $239) will cost the participant $402 and will incur a cost of $194 against the participant's total available frame benefit of $194 and a cost of $95 against the participant's total available lens benefit of $114. As displayed in section 915, the participant cost of $402 represents a total savings of $331 over the retail cost of the combination of these options ($733). The system may close the sale and complete the purchase process in response to detecting the participant's selection of the control 962.

In various embodiments, the system may facilitate the purchase of multiple eye care-related products and/or services. For example, a participant may be eligible, under the participant's vision plan, to receive an eye exam and two pairs of glasses every two years. In another example, a participant may be covered under more than one plan. Alternatively, the participant may simply desire to purchase additional products and/or services regardless of whether such additional products and/or services are fully covered under the participant's vision plan. As shown in FIG. 9, the interface 900 may facilitate the selection of a subsequent product or service by providing a control 964 that allows the participant to add another product or service (e.g., in FIG. 9 “add another pair” of glasses).

In response to the selection of a control, such as the control 964 of interface 900, the system may generate an interface similar to any of those described herein to obtain participant selections for additional eye care-related products and/or services. For example, the system may return the participant to an initial interface, such as the interface 500 of FIG. 5, where the participant can select a different type of product or service and begin the purchase transaction process for that product or service. The system may also allow the participant to select a different vision plan to use with the purchase of the subsequent product or service. Such selections may result in the configuration of, for example, additional eyewear.

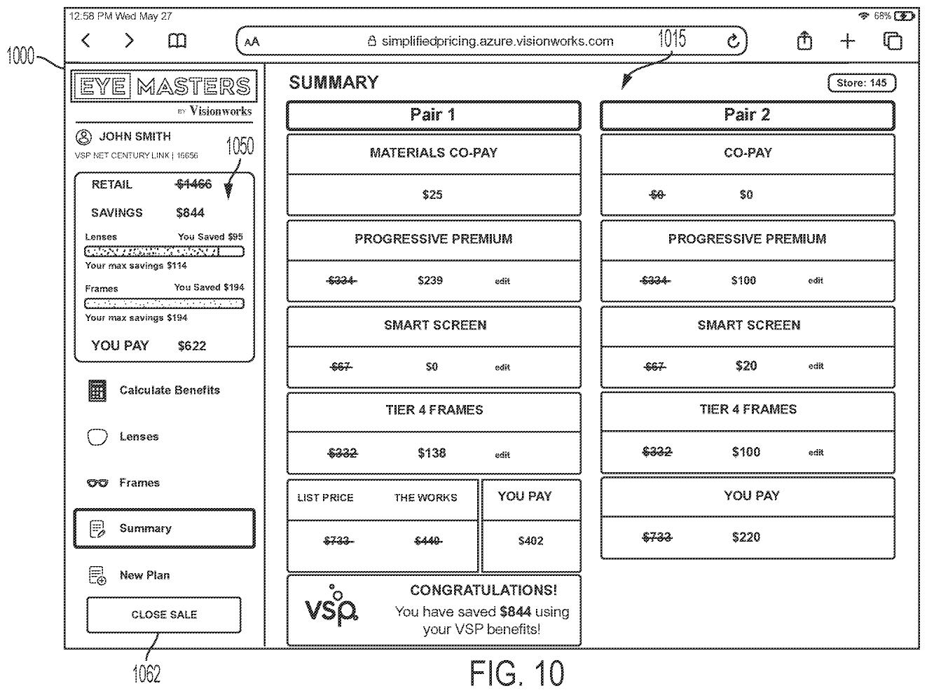

FIG. 10 illustrates another example interface 1000 that the system (e.g., the Eye Care Transaction Server 110) may present to a participant summarizing the selection of multiple eye care products and/or services. After receiving one or more selections and/or information from the participant associated with the purchase of each of such multiple eye care products and/or services (e.g., at interfaces such as those described in regard to FIGS. 5-8), the system may generate the interface 1000 to present a summary of the options selected by the participant for each product or service and the total cost, benefit, and savings. The system may present the summary section 1015 displaying the selected products and/or services and associated options, which in FIG. 10 represents two pairs of glasses, each with progressive premium lenses, various lens options, and Tier 4 frames, continuing with the example used in regard to the previously described interfaces. Also shown in the section 1015 is the retail cost associated with each item and the respective participant cost, representing the savings the participant achieved using the vision plan. In particular embodiments, products summarized in such an interface may be covered by different vision plans. In such embodiments, the summary section 1015 may also display the vision plan associated with the purchase of each of the selected products and/or services and associated options.

The system may present the final plan information section 1050 for the purchase of multiple items that includes a summary of the benefits available to the participant, the total benefits used for the particular product or service, and the total cost to participant for the particular product or service based on the participant's selections. For example, in the exemplary interface shown in FIG. 10, and continuing with the example used in regard to the previous interfaces, the participant's various selections resulted in a total cost to the participant of $622 and used a total of $194 against the participant's total available frame benefit of $194 and used a total of $95 against the participant's total available lens benefit of $114. As displayed in the section 1015, the participant cost of $622 represents a total savings of $844 over the retail cost of these two sets of eyewear ($733 each, for a total retail cost of $1,466). The system may close the sale complete the purchase process in response to detecting the participant's selected of the control 1062.

As noted above, the system may use machine learning (e.g., in conjunction with big data processing techniques) to improve the participant's user experience and/or to accomplish any of a variety of goals, such as maximizing the participant's use of their benefits, educating the participant regarding their available benefits, maximizing profitability, maximizing the sale of one or more particular brands and/or types of eyewear products (e.g., frames, lenses, or coatings). In doing so, the system may, for example: (1) access a data structure that includes detailed data from a large number of past eyewear purchase transactions facilitated by the system (e.g., the type and/or brand of eyewear purchased, the type of lenses purchased, the type and/or brand of any lens coatings purchased, purchase price information for the transaction, how much (what percentage) of the participant's benefits were used during the transaction, etc.); (2) use artificial intelligence to analyze data from the data structure to identify one or more patterns that indicate that modifying the operation of the system in a particular way would improving progress towards achieving a particular goal (e.g., any of the goals listed above); and (3) automatically modifying the operation of the system in the particular way.

In particular embodiments, modifying the operation of the system in the example described above may involve, for example, during any one or more of the steps executed by the system: (1) changing an order in which one or more types of information are displayed; (2) changing the wording of a particular offer; (3) displaying an offer of a particular type or level of discount; (4) displaying less or more information regarding the transaction or the participant's benefits; (5) modifying a format of information displayed by the system; and/or (6) displaying information in a particular font.

### Conclusion

Although embodiments above are described in reference to various systems and methods for calculating cost and benefit parameters for an eye care-related purchase transaction, it should be understood that various aspects of the systems and methods described above may be applicable to other health care-related systems, purchasing systems, or to other types of systems, in general. For example, the functionality described above for obtaining participant selections of various options related to a product or service and the calculation and presentation of costs, benefits, and savings based on a vision plan may be used within the context of any system that provides defined benefits that can be applied to the purchase of any suitable goods or services.

While this specification contains many specific embodiment details, these should not be construed as limitations on the scope of any invention or of what may be claimed, but rather as descriptions of features that may be specific to particular embodiments of particular inventions. Certain features that are described in this specification in the context of separate embodiments may also be implemented in combination in a single embodiment. Conversely, various features that are described in the context of a single embodiment may also be implemented in multiple embodiments separately and/or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products.

Many modifications and other embodiments of the invention will come to mind to one skilled in the art to which this invention pertains having the benefit of the teachings presented in the foregoing descriptions and the associated drawings. While examples discussed above cover the use of various embodiments in the context of calculating cost and benefit parameters for an eye care-related purchase transaction, various embodiments may be used in any other suitable context. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for the purposes of limitation.

What is claimed is:

1. A data processing system enhancing participant experience during an electronic eyewear purchasing transaction, the data processing system comprising:
at least one computer processor; and
memory operatively coupled to the at least one computer processor and storing computer-readable instructions, wherein the at least one computer processor is adapted for:
executing the computer-readable instructions to perform operations comprising:
electronically receiving a request from a participant to initiate the electronic eyewear purchasing transaction via a first graphical user interface displayed on a video display unit, the request comprising participant identification data and optical option plan identification data;
at least partially in response to receiving the request:
identifying a particular optical option plan data structure based at least in part on the optical option plan identification data;
generating an interaction data structure configured for representing the eyewear product purchase transaction associated with the participant;
populating a plurality of fields of the interaction data structure using the participant identification data and the optical option plan identification data;
populating an interaction status field in the interaction data structure with a first status representing the eyewear product purchase transaction has been initialized;
determining a total available benefit value based at least in part on the particular optical option plan data structure; and
determining a plurality of first optical options based at least in part on the particular optical option plan data structure;
processing the interaction data structure using a preferred options machine learning model to generate a prediction of a preference of the participant for each first optical option of the plurality of first optical options;
generating a second graphical user interface by configuring the second graphical user interface to comprise at least one first selectable control displayed on the second graphical user interface to provide the plurality of first optical options configured in a manner to enhance selection by the participant based at least in part on the prediction of the preference of the participant for each first optical option of the plurality of first optical options;
providing the second graphical user interface for display on the video display unit for receiving a selection of a first optical option by the participant via the at least one first selectable control from among the plurality of first optical options, wherein the second graphical user interface is configured to:
display an indication of a respective participant cost for each first optical option of the first plurality of first optical options;
display an indication of a respective benefit value for each first optical option of the first plurality of first optical options; and
display an indication of the total available benefit value;
electronically receiving the selection of the first optical option via the second graphical user interface; and
responsive to receiving the selection of the first optical option:
updating the interaction status field in the interaction data structure based at least in part on the selection of the first optical option; and
updating one or more fields in the interaction data structure to reflect the selection of the first optical option;
determining an adjusted total available benefit value based at least in part on the selection of the first optical option;
determining a plurality of second optical options based at least in part on the particular optical option plan data structure and the selection of the first optical option;
processing the interaction data structure using the preferred options machine learning model to generate a prediction of a preference of the participant for each second optical option of the plurality of second optical options;
generating a third graphical user interface by configuring the third graphical user interface to comprise at least one second selectable control displayed on the third graphical user interface to provide the plurality of second optical options configured in a manner to enhance selection by the participant based at least in part on the prediction of the preference of the participant for each second optical option of the plurality of second optical options; and
providing the third graphical user interface for display on the video display unit for receiving a selection of a second optical option by the participant via the at least one second selectable control from among the plurality of second optical options, wherein the second graphical user interface is configured to:
display an indication of a respective participant cost for each first optical option of the plurality of first optical options;
display an indication of a respective benefit value for each first optical option of the plurality of first optical options; and
display an indication of the total available benefit value.

2. The data processing system of claim 1, wherein one or more of the plurality of first optical options comprises at least one of:
(a) contact lenses;
(b) glasses;
(c) lenses;
(d) frames;
(e) lens coatings; or
(f) an eye exam.

3. (canceled)

4. The data processing system of claim 1, wherein the operations further comprise:
determining a benefit value for the first optical option based at least in part on the optical option plan data structure;
determining a participant cost for the first optical option based at least in part on the optical option plan data structure; and
updating the second graphical user interface to:
display the first optical option;
display an indication of the benefit value for the first optical option; and
display an indication of the participant cost for the first optical option.

5. The data processing system of claim 1, wherein:
the total available benefit value is a total benefit value for a particular component of the first optical option; and
the particular component of the first optical option comprises at least one of:
(a) lenses;
(b) frames; or
(c) contact lenses.

6. The data processing system of claim 1, wherein the operations further comprise updating the third graphical user interface to display an indication of an interaction status based at least in part on the updated interaction status field in the interaction data structure.

7. A non-transitory computer-readable medium storing computer-executable instructions for:
receiving an indication that an individual has selected one or more vision products for purchase;
at least partially in response to receiving the indication, accessing from one or more data stores, data regarding a vision plan held by the individual covering at least one of the one or more vision products;
receiving a listing of the one or more selected vision products; and
based at least in part on the received listing and the accessed vision plan data, communicating to the individual, via a computer interface:
(1) an amount of savings that they will receive if they complete a purchase of the one or more vision products; and
(2) an indication of benefits, under the vision plan, that will go unused if the individual completes the purchase without adding additional products to the purchase or substituting one or more of the vision products with another item.

8. The non-transitory computer-readable medium of claim 7, wherein accessing the data regarding the vision plan held by the individual comprises identifying the vision plan held by the individual from among a plurality of vision plans based on identification information associated with the individual.

9. (canceled)

10. The non-transitory computer-readable medium of claim 8, wherein:
a first one or more plans of the plurality of vision plans is associated with a first particular provider;
a second one or more plans of the plurality of vision plans is associated with a second particular provider; and
the first provider is distinct from the second provider.

11. The non-transitory computer-readable medium of claim 7, wherein accessing the data regarding the vision plan held by the individual comprises identifying the vision plan from among a plurality of vision plans held by the individual.

12. The non-transitory computer-readable medium of claim 11, further comprising computer-executable instructions for:
identifying a second vision plan from among the plurality of vision plans held by the individual;
accessing data regarding the second vision plan held by the individual covering at least one of the one or more vision products; and
based at least in part on the received listing and the accessed second vision plan data, communicating to the individual, via the computer interface:
(1) a second amount of savings associated with a purchase of the one or more vision products under the second vision plan; and
(2) an indication of benefits, under the second vision plan, that will go unused if the individual completes the purchase of the one or more vision products under the second vision plan without adding additional products to a purchase transaction associated with the purchase of the one or more vision products under the second vision plan.

13. The non-transitory computer-readable medium of claim 7, wherein the additional products comprise one or more products selected from a group consisting of:
(a) a lens coating;
(b) lens polarization;
(c) lens light reactivity;
(d) a particular lens thinness; and
(e) lens magnification progressivity.

14. A computer-implemented data processing method for enhancing participant experience during an electronic eyewear purchasing transaction, the method comprising:
receiving, by one or more computer processors, participant data for a participant originating from one or more participant data inputs provided by a participant on a first graphical user interface displayed on a video display unit;
retrieving, by the one or more computer processors from a data repository, based at least in part on the participant data, plan data for a particular vision plan;
generating, by the one or more computer processors, a transaction data structure configured for representing the electronic eyewear purchasing transaction associated with the participant;
populating, by the one or more computer processors, a plurality of data fields in the transaction data structure using the plan data and the participant data;
populating, by the one or more computer processors, a transaction status field in the transaction data structure with a first status representing the electronic eyewear purchasing transaction has been initialized;
determining, by the one or more computer processors, based at least in part on the plan data, (i) a total available benefit value, (ii) a plurality of first eyewear product options, (iii) a respective participant cost for each first eyewear product option of the plurality of first eyewear product options, and (iv) a respective benefit value for each first eyewear product option of the plurality of first eyewear product options;
processing, by the one or more computer processors, the transaction data structure using a preferred options machine learning model to generate a prediction of a preference of the participant for each first eyewear product option of the plurality of first eyewear product options;
generating, by the one or more computer processors, a second graphical user interface by configuring the second graphical user interface to comprise at least one first selectable control displayed on the second graphical user interface to provide the plurality of first eyewear product options configured in a manner to enhance selection by the participant based at least in part on the prediction of the preference of the participant for each first eyewear product option of the plurality of first eyewear product options;
providing, by the one or more computer processors, the second graphical user interface for display on the video display unit for receiving a selection of a first eyewear product option by the participant via the at least one first selectable control from among the plurality of first eyewear product options, wherein the second graphical user interface further displays:
an indication of the respective participant cost for each first eyewear product option of the plurality of first eyewear product options;
an indication of the respective benefit value for each first eyewear product option of the plurality of first eyewear product options; and
an indication of the total available benefit value;
receiving, by the one or more computer processors, the selection of the first eyewear product option originating from the second graphical user interface; and
responsive to receiving the selection of the first eyewear product option:
populating, by the one or more computer processors, the transaction status field in the transaction data structure with a second status reflecting the selection of the first eyewear product option;
determining, by the one or more computer processors, based at least in part on the selection of the first eyewear product option and the plan data, (i) a participant cost for the first eyewear product option, (ii) an adjusted total available benefit value, and (iii) a plurality of second eyewear product options;
determining, by the one or more computer processors, based at least in part on the plan data, (i) a respective participant cost for each second eyewear product option of the second plurality of second eyewear product options and (ii) a respective benefit value for each second eyewear product option of the plurality of second eyewear product options;
populating, by the one or more computer processors, at least one data field in the transaction data structure with the selection of the first eyewear product option;
after populating the at least one data field in the transaction data structure with the selection of the first eyewear product option, processing, by the one or more computer processors, the transaction data structure using the preferred options machine learning model to generate a prediction of a preference of the participant for each second eyewear product option of the plurality of second eyewear product options;
generating, by the one or more computer processors, a third graphical user interface by configuring the third graphical user interface to comprise at least one second selectable control displayed on the third graphical user interface to provide the plurality of second eyewear product options configured in a manner to enhance selection by the participant based at least in part on the prediction of the preference of the participant for each second eyewear product option of the plurality of second eyewear product options; and
providing, by the one or more computer processors, the third graphical user interface for display on the video display unit for receiving a selection of a second eyewear product option by the participant via the at least one second selectable control from among the plurality of second eyewear product options, wherein the third graphical user interface further display:
an indication of the respective participant cost for each second eyewear product option of the plurality of second eyewear product options;
an indication of the respective benefit value for each second eyewear product option of the plurality of second eyewear product options;
an indication of the participant cost for the first eyewear product option; and
an indication of the adjusted total available benefit value.

15. The computer-implemented data processing method of claim 14, wherein:
the method further comprises determining, by the one or more computer processors, based at least in part on the plan data, a respective retail price for each first eyewear product option of the first plurality of first eyewear product options, wherein;
the second graphical user interface further displays an indication of the respective retail price for each first eyewear product option of the plurality of first eyewear product options.

16. The computer-implemented data processing method of claim 14, wherein the third graphical user interface further displays the indication of the total available benefit value.

17. The computer-implemented data processing method of claim 14 further comprising populating, by the one or more computer processors, based at least in part on the selection of the first eyewear product option, one or more additional data fields in the transaction data structure.

18. The computer-implemented data processing method of claim 14, wherein:
each of the plurality of first eyewear product options is a lens option; and
each of the plurality of second eyewear product options is a frame option.

19. The computer-implemented data processing method of claim 14 further comprising populating, by the one or more computer processors, based at least in part on the selection of the first eyewear product option, a data field in the transaction data structure with a benefit value associated with the first eyewear product option.

20. The computer-implemented data processing method of claim 14 further comprising populating, by the one or more computer processors, based at least in part on the selection of the first eyewear product option, a data field in the transaction data structure with a participant cost associated with the first eyewear product option.

21. A non-transitory computer-readable medium storing computer-executable instructions configured to, when executed by one or more computer processors, cause the one or more computer processors to:
electronically receive a request from a participant to initiate an electronic eyewear purchasing transaction via a first graphical user interface displayed on a video display unit, the request comprising participant identification data and optical option plan identification data;
at least partially in response to receiving the request:
identify a particular optical option plan data structure based at least in part on the optical option plan identification data;
generate an interaction data structure configured for representing the electronic eyewear purchasing transaction associated with the participant;
populate a plurality of fields of the interaction data structure using the participant identification data and the optical option plan identification data;
populate an interaction status field in the interaction data structure with a first status representing the electronic eyewear purchasing transaction has been initialized;
determine a total available benefit value based at least in part on the particular optical option plan data structure; and
determine a first plurality of first optical options based at least in part on the particular optical option plan data structure;
process the interaction data structure using a preferred options machine learning model to generate a prediction of a preference of the participant for each first optical option of the plurality of first optical options;
generate a second graphical user interface by configuring the second graphical user interface to comprise at least one first selectable control displayed on the second graphical user interface to provide the plurality of first optical options configured in a manner to enhance selection by the participant based at least in part on the prediction of the preference of the participant for each first optical option of the plurality of first optical options;
provide the second graphical user interface for display on the video display unit for receiving a selection of a first optical option by the participant via the at least one first selectable control from among the plurality of first optical options, wherein the second graphical user interface is configured to:
display an indication of a respective participant cost for each first optical option of the plurality of first optical options;
display an indication of a respective benefit value for each first optical option of the first plurality of first optical options; and
display an indication of the total available benefit value;
electronically receive the selection of the first optical option via the second graphical user interface; and
responsive to receiving the selection of the first optical option:
update the interaction status field in the interaction data structure based at least in part on the selection of the first optical option; and
update one or more fields in the interaction data structure to reflect the selection of the first optical option;
determine an adjusted total available benefit value based at least in part on the selection of the first optical option;
determine a plurality of second optical options based at least in part on the particular optical option plan data structure and the selection of the first optical option;
process the interaction data structure using the preferred options machine learning model to generate a prediction of a preference of the participant for each second optical option of the plurality of second optical options;
generate a third graphical user interface by configuring the third graphical user interface to comprise at least one second selectable control displayed on the third graphical user interface to provide the plurality of second optical options configured in a manner to enhance selection by the participant based at least in part on the prediction of the preference of the participant for each second optical option of the plurality of second optical options; and
provide the third graphical user interface for display on the video display unit for receiving a selection of a second optical option by the participant via the at least one second selectable control from among the plurality of second optical options, wherein the second graphical user interface is configured to:
display an indication of a respective participant cost for each first optical option of the plurality of first optical options;
display an indication of a respective benefit value for each first optical option of the plurality of first optical options; and
display an indication of the total available benefit value.

22. The non-transitory computer-readable medium of claim 21, wherein the computer-executable instructions are configured to, when executed by the one or more computer processors, cause the one or more computer processors to:
determine a benefit value for the first optical option based at least in part on the optical option plan data structure;
determine a participant cost for the first optical option based at least in part on the optical option plan data structure; and
update the second graphical user interface to:
display the first optical option;
display an indication of the benefit value for the first optical option; and
display an indication of the participant cost for the first optical option.

23. (canceled)

24. The non-transitory computer-readable medium of claim 21, wherein the computer-executable instructions are configured to, when executed by the one or more computer processors, cause the one or more computer processors to update the third graphical user interface to display an indication of an interaction status based at least in part on the updated interaction status field in the interaction data structure.

25. The data processing system of claim 1, wherein configuring the plurality of first eyewear product options in the manner to enhance selection by the participant based at least in part on the prediction of the preference of the participant for each first optical option of the plurality of first optical options comprises at least one of configuring the plurality of first optical options to:
display in a particular order;
display only the first optical options from the plurality of first optical options for a particular type of optical option;
display at least one first optical option of the plurality of first optical options with changed wording than normally displayed;
display at least one first optical option of the plurality of first optical options with additional information than normally displayed; or
display at least one first optical option of the plurality of first optical options in a different font than normally displayed.

26. The computer-implemented data processing method of claim 14, wherein configuring the plurality of first eyewear product options in the manner to enhance selection by the participant based at least in part on the prediction of the preference of the participant for each first eyewear product option of the plurality of first eyewear product options comprises at least one of configuring the plurality of first eyewear product options to:
display in a particular order;
display only the first eyewear product options from the plurality of first eyewear product options for a particular type of eyewear product option;
display at least one first eyewear product option of the plurality of first eyewear product options with changed wording than normally displayed;
display at least one first eyewear product option of the plurality of first eyewear product options with additional information than normally displayed; or
display at least one first eyewear product option of the plurality of first eyewear product options in a different font than normally displayed.

27. The non-transitory computer-readable medium of claim 21, wherein configuring the plurality of first eyewear product options in the manner to enhance selection by the participant based at least in part on the prediction of the preference of the participant for each first optical option of the plurality of first optical options comprises at least one of configuring the plurality of first optical options to:
display in a particular order;
display only the first optical options from the plurality of first optical options for a particular type of optical option;
display at least one first optical option of the plurality of first optical options with changed wording than normally displayed;
display at least one first optical option of the plurality of first optical options with additional information than normally displayed; or
display at least one first optical option of the plurality of first optical options in a different font than normally displayed.
